(19) United States
(12) Patent Application Publication
(10) Pub. No.: US 2010/0209452 A1
(43) Pub. Date: Aug. 19, 2010

(54) COMPOSITIONS COMPRISING AN ANTIGEN, AN AMPHIPATHIC COMPOUND AND A HYDROPHOBIC CARRIER, AND USES THEREOF

(75) Inventor: Marc Mansour, Halifax (CA)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(73) Assignee: IMMUNOVACCINE TECHNOLOGIES, INC, Halifax, Nova Scotia (CA)

(21) Appl. No.: 12/679,998

(22) PCT Filed: Oct. 2, 2008
(86) PCT No.: PCT/CA08/01747

§ 371 Date: Mar. 25, 2010

Related U.S. Application Data
(60) Provisional application No. 60/977,192, filed on Oct. 3, 2007.

(51) Int. Cl. A61K 39/12; A61K 39/00; A61K 39/02; A61P 37/04

(52) U.S. Cl. 424/204.1; 424/184.1; 424/234.1

(57) ABSTRACT

The present invention provides compositions comprising an antigen, an amphipathic compound, and a hydrophobic carrier and methods of using these compositions for inducing an antibody or cell-mediated response in a subject.

### CROSS-REFERENCE TO RELATED APPLICATIONS

This application claims the benefit of and priority from U.S. Provisional Patent Application No. 60/977,197, filed Oct. 3, 2007, which is incorporated herein by reference in its entirety.

### FIELD OF THE INVENTION

The invention concerns compositions comprising an antigen, an amphipathic compound, and a hydrophobic carrier. Compositions of the invention have been found to provide enhanced immune responses in vivo.

### BACKGROUND OF THE INVENTION

Vaccination generally involves the injection of an antigenic substance or antigen into an animal. The antigenic substance generates an immune response in the animal. The antigen may be e.g. a killed organism such as a bacterium or an inactivated virus, a component of an organism that has antigenic properties, a live organism or virus with low virulence.

The effectiveness of an antigen in stimulating an immune response can be enhanced by administration with an adjuvant. An adjuvant may function by different mechanisms, including (1) trapping the antigen in the body to cause a slow release, (2) attracting cells of the immune system to the injection site, (3) stimulating cells of the immune system to proliferate and to become activated, and (4) improving antigen dispersion in the recipient's body. Commonly used adjuvants include aluminum salts, water-in-oil and oil-in water emulsions, mineral salts and other compounds that can act as a stimulatory danger signal. Polycations such as diethylaminoethyldextran (DEAE dextran) may also be effective as adjuvants in some cases. The adjuvant may be included in the vaccine as an additive or may be administered separately.

Many vaccine compositions are water-in-oil or oil-in-water emulsions. Water-in-oil compositions in particular are effective because of the prolonged presence of such compositions at the injection site, causing the slow release of antigen at the site of immunization. However, water-in-oil emulsions may become unstable once injected in vivo, causing the separation of the aqueous and oily phases of the composition. This leads to premature or accelerated release of antigens and other components.

### SUMMARY OF THE INVENTION

In one aspect, the invention provides a composition comprising: an antigen; an amphipathic compound; and a hydrophobic carrier; wherein the composition is substantially free of water.

In another aspect, the invention provides a process for making the composition as described above, the process comprising: (a) combining an antigen and an amphipathic compound to form a dry mixture; and (b) suspending said mixture in a hydrophobic carrier; wherein the composition is substantially free of water.

In another aspect, the invention provides a method comprising administering the composition as described above to a subject.

In another aspect, the invention provides a method for inducing an antibody response or cell-mediated immune response in a subject, the method comprising administering to a subject in need thereof the composition as described above.

### BRIEF DESCRIPTION OF THE DRAWINGS

FIG. 1 illustrates tumor growth over a 42-day period in mice implanted with C3 cells and treated with phosphate buffered saline 8 days post tumor implantation according to the present invention. Control mice (group 1, n=10) implanted with C3 cells exhibited normal tumor growth over a period of 42 days. All mice in this group were treated with phosphate buffered saline 8 days post tumor implantation (PTI).

FIG. 2 illustrates tumor growth over a 42-day period in mice implanted with C3 cells and treated on day 8 with a control formulation consisting of FP antigen in a water-in-oil emulsion. Control mice (group 2, n=8) were implanted with C3 cells and treated on day 8 with a control formulation consisting of FP antigen in a water-in-oil emulsion. Tumors were monitored weekly for a total of 42 days.

FIG. 3 illustrates tumor growth over a 42-day period in mice implanted with C3 cells and treated on day 8 with a water-free composition consisting of FP antigen, DOPC carrier and a hydrophobic carrier (Incomplete Freund's adjuvant). Mice (group 3, n=8) were implanted with C3 cells and treated on day 8 with a water-free composition consisting of FP antigen, DOPC carrier and a hydrophobic carrier (Incomplete Freund's adjuvant). Tumors were monitored weekly for a total of 42 days.

FIG. 4 illustrates tumor growth in mice implanted with C3 cells and treated on day 8 with a control formulation consisting of FP antigen and Pam3Cys adjuvant in a water-in-oil emulsion (incomplete Freund's adjuvant). Control mice (group 4, n=8) implanted with C3 cells and treated on day 8 with a control formulation consisting of FP antigen and Pam3Cys adjuvant in a water-in-oil emulsion (incomplete Freund's adjuvant). Tumors were monitored weekly for a total of 42 days.

FIG. 5 illustrates tumor growth over a 42-day period in mice implanted with C3 cells and treated on day 8 with a water-free composition consisting of FP antigen, Pam3Cys adjuvant, DOPC carrier and a hydrophobic carrier (Incomplete Freund's adjuvant). Mice (group 5, n=7) implanted with C3 cells and treated on day 8 with a water-free composition consisting of FP antigen, Pam3Cys adjuvant, DOPC carrier and a hydrophobic carrier (Incomplete Freund's adjuvant). Tumors were monitored weekly for a total of 42 days.

FIG. 6 illustrates cellular immune response in three groups of mice (n=4) vaccinated as follows: group 1 mice were vaccinated with FP in a typical water-in-oil emulsion (incomplete Freund's adjuvant), group 2 mice were vaccinated with a water-free formulation consisting of FP, DOPC and incomplete Freund's adjuvant, and group 3 mice were vaccinated with a control water-free formulation consisting of FP and incomplete Freund's adjuvant. Cellular immune responses were measured by ELISPOT assay and are presented as an average of spot forming units.

FIG. 7 shows vials (front elevation) containing hydrophobic carrier (vial ISA51), polyIC formulated according to the invention (vial 21), and polyIC suspended in the hydrophobic carrier in the absence of the amphipathic compound DOPC (vial 26). A heterogeneous suspension of insoluble polyIC strands can be easily seen in vial 26.

FIG. 8 shows vials (bottom plan view) containing hydrophobic carrier (vial ISA51), peptide antigens formulated according to the invention (vial 30), and peptide antigens suspended in the hydrophobic carrier in the absence of the amphipathic compound DOPC (vial 35). Antigen aggregates that could not resuspended in the hydrophobic carrier can be easily seen in vial 35 (circled).

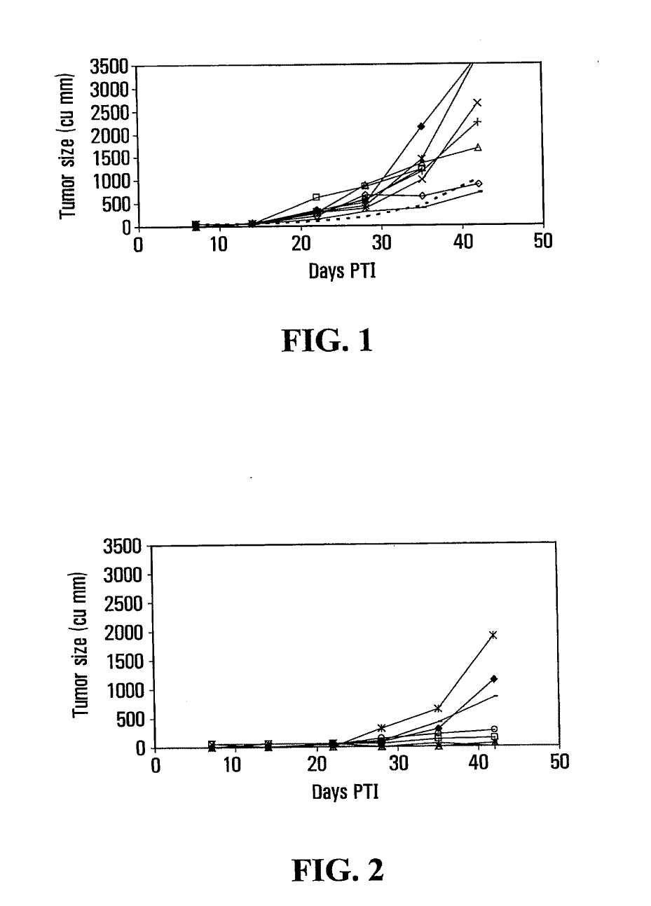

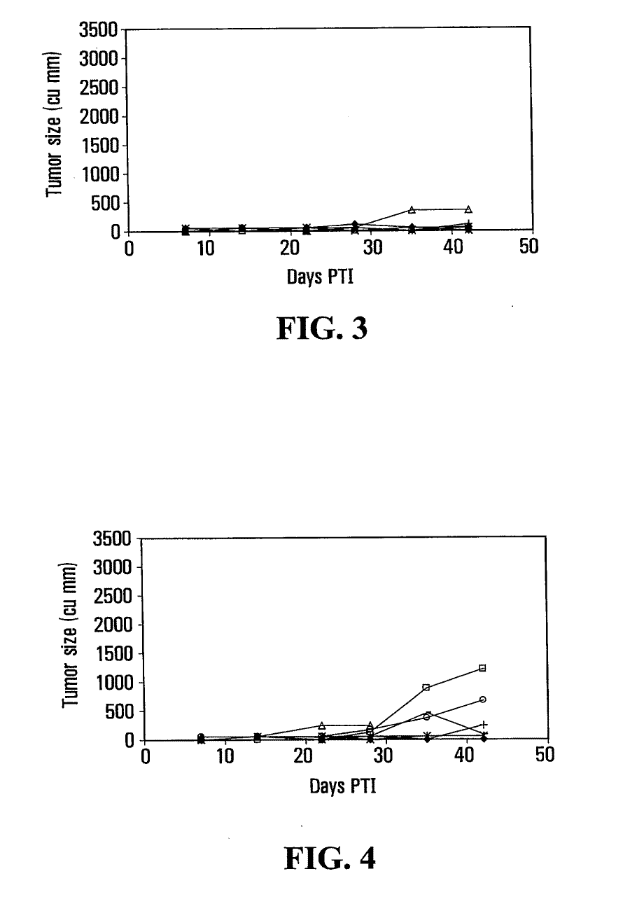

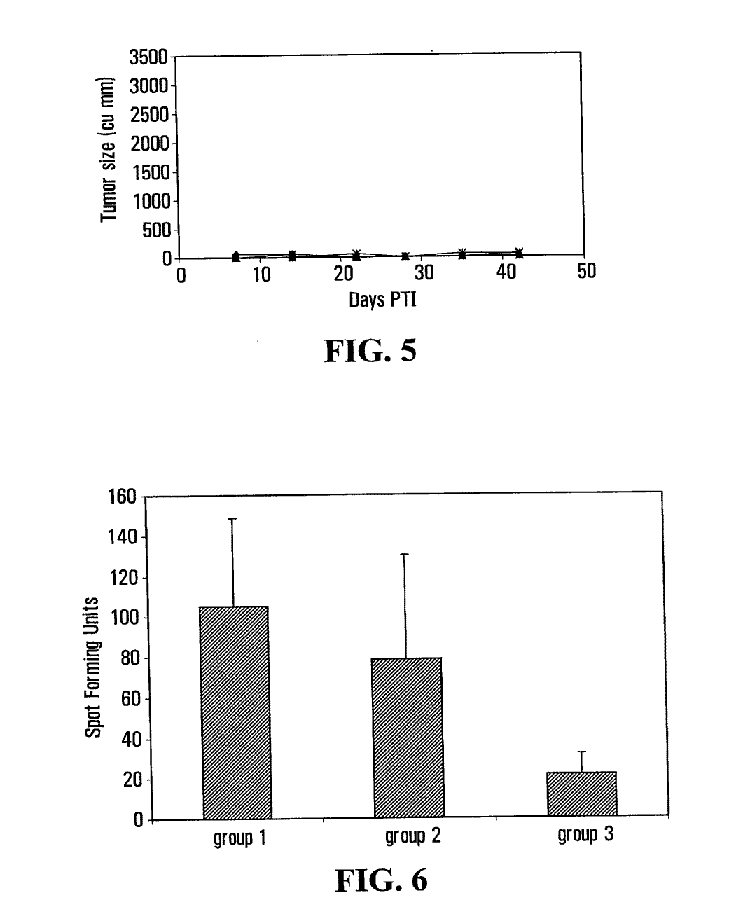

### DETAILED DESCRIPTION

The invention provides compositions comprising, consisting essentially of, or consisting of: an antigen; an amphipathic compound; and a hydrophobic carrier; wherein the composition is substantially free of water.

### Antigens

The compositions of the invention comprise one or more antigens. As used herein, the term “antigen” refers to a substance that can bind specifically to an antibody or to a T-cell receptor.

Antigens useful in the compositions of the invention, include, without limitation, polypeptides, a microorganism or a part thereof, such as a live, attenuated, inactivated or killed bacterium, virus or protozoan, or part thereof.

As used herein and in the claims, the term “antigen” also includes a polynucleotide that encodes the polypeptide that functions as an antigen. Nucleic acid-based vaccination strategies are known, wherein a vaccine composition that contains a polynucleotide is administered to a subject. The antigenic polypeptide encoded by the polynucleotide is expressed in the subject, such that the antigenic polypeptide is ultimately present in the subject, just as if the vaccine composition itself had contained the polypeptide. For the purposes of the present invention, the term “antigen”, where the context dictates, encompasses such polynucleotides that encode the polypeptide which functions as the antigen.

Polypeptides or fragments thereof that may be useful as antigens in the invention include, without limitation, those derived from Cholera toxoid, tetanus toxoid, diphtheria toxoid, hepatitis B surface antigen, hemagglutinin, neuraminidase, influenza M protein, PfHRP2, pLDH, aldolase, MSP1, MSP2, AMA1, Der-p-1, Der-f-1, Adipophilin, AFP, AIM-2, ART-4, BAGE, alpha-fetoprotein, BCL-2, Bcr-Abl, BING-4, CEA, CPSF, CT, cyclin D1Ep-CAM, EphA2, EphA3, ELF-2, FGF-5, G250, Gonadotropin Releasing Hormone, HER-2, intestinal carboxyl esterase (iCE), IL13Ralpha2, MAGE-1, MAGE-2, MAGE-3, MART-1, MART-2, M-CSF, MDM-2, MMP-2, MUC-1, NY-EOS-1, MUM-1, MUM-2, MUM-3, p53, PBF, PRAME, PSA, PSMA, RAGE-1, RNF43, RU1, RU2AS, SART-1, SART-2, SART-3, SAGE-1, SCRN 1, SOX2, SOX10, STEAP1, surviving, Telomerase, TGFbetaRII, TRAG-3, TRP-1, TRP-2, TERT and WT1.

Viruses, or parts thereof, useful as antigens in the invention include, without limitation, Cowpoxvirus, Vaccinia virus, Pseudocowpox virus, Human herpesvirus 1, Human herpesvirus 2, Cytomegalovirus, Human adenovirus A-F, Polyomavirus, Human papillomavirus, Parvovirus, Hepatitis A virus, Hepatitis B virus, Hepatitis C virus, Human immunodeficiency virus, Orthoreovirus, Rotavirus, Ebolavirus, parainfluenza virus, influenza A virus, influenza B virus, influenza C virus, Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella.

Bacteria or parts of thereof useful as antigens in the invention include, without limitation, Anthrax, Brucella, Candida, Chlamydia pneumoniae, Chlamydia psittaci, Cholera, Clostridium botulinum, Coccidioides immitis, Cryptococcus, Diphtheria, Escherichia coli 0157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica.

The antigen may alternatively be of protozoan origin, e.g. Plasmodium falciparum, which causes malaria.

As used herein, the term “polypeptide” or “protein” means any chain of amino acids, regardless of length (e.g. 4, 6, 8, 10, 20, 50, 100, 200, 500 or more amino acids) or post-translational modification (e.g., glycosylation or phosphorylation). Both terms are used interchangeably. The terms “polypeptide” and “protein” are intended to encompass molecules (such as peptidomimetics) mimicking the properties or function of a polypeptide or protein but incorporating modifications to alter the molecule's properties, such as the molecule's stability or biological activity. These modifications include e.g. altered backbones (e.g. inclusion of non-peptidic bonds) and the incorporation of non-naturally occurring amino acids.

As used herein the term “polynucleotide” encompasses a chain of nucleotides of any length (e.g. 9, 12, 18, 24, 30, 60, 150, 300, 600, 1500 or more nucleotides) or number of strands (e.g. single-stranded or double-stranded). Polynucleotides may be DNA (e.g. genomic DNA or cDNA) or RNA (e.g. mRNA) or combinations thereof. They may be naturally occurring or synthetic (e.g. chemically synthesized). It is contemplated that the polynucleotide may contain modifications of one or more nitrogenous bases, pentose sugars or phosphate groups in the nucleotide chain. Such modifications are well-known in the art and may be for the purpose of e.g. improving stability of the polynucleotide.

The concentration of antigen may be as high as required to effectively stimulate an immune response, with the limitations on the amount of antigen being that the antigen should not precipitate out of the composition, and the antigen must be re-suspendable into the hydrophobic carrier. Further, the concentration of antigen varies depending on the type of antigen and the amount of other components in the composition. One skilled in the art can readily determine the amount of antigen needed in a particular application. For example, for peptide antigens about 0.01 to about 5 mg/ml may be used (based on the total volume of the composition), with the preferred range being not less than 0.1 and not more than 1.0 mg/ml. For other antigens, such as recombinant proteins, the concentration may be in the range of about 0.01 to about 0.5 mg/ml, with the preferred range being not less than 0.01 and not more than 0.5 mg/ml.

### Amphipathic Compounds

The compositions of the invention comprise one or more amphipathic compounds. An “amphipathic compound” is a compound having both hydrophilic and hydrophobic parts or characteristics. The hydrophobic portion of an amphipathic compound is typically a large hydrocarbon moiety, such as a long chain of the form CH₃(CH₂)ₙ, with n>4. The hydrophilic portion of an amphipathic compound is usually either a charged group or a polar uncharged group. Charged groups include anionic and cationic groups. Examples of anionic charged groups include the following (wherein the hydrophobic part of the molecule is represented by “R”): carboxylates: RCO₂⁻; sulfates: RSO₄⁻; sulfonates: RSO₃⁻; and phosphates (the charged functionality in phospholipids). Cationic charged groups include e.g. amines: RNH₃⁺ (“R” again representing the hydrophobic part of the molecule). Uncharged polar groups include e.g. alcohols with large R groups, such as diacyl glycerol (DAG). Amphipathic compounds may have several hydrophobic parts, several hydrophilic parts, or several of both. Proteins and some block copolymers are examples. Steroids, cholesterol, fatty acids, bile acids, and saponins, are also amphipathic compounds useful in the practice of the invention.

The compositions of the invention may contain a single amphipathic compound or a mixture of amphipathic compounds. In some embodiments, the amphipathic compound(s) is a phospholipid or mixture of phospholipids.

Broadly defined, a “phospholipid” is a member of a group of lipid compounds that yield on hydrolysis phosphoric acid, an alcohol, fatty acid, and nitrogenous base. Phospholipids that may be used in the practice of the invention, include phosphoglycerides, which are phospholipids in which two fatty acyl side chains are esterified to two of the three hydroxyl groups of a glycerol molecule. The third hydroxyl group of the glycerol molecule is esterified with phosphate. The phosphate group is usually also esterified to a hydroxyl group on a hydrophilic compound such as ethanolamine, serine, choline, or glycerol. Phospholipids that are phosphoglycerides include, e.g. phosphatidylethanolamine, phosphatidylserine, phosphatidylcholine, dioleoyl phosphatidylcholine (“DOPC”), phosphatidylinositol, and diphosphatidylglycerol. Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic. All of these and other phospholipids may be used in the practice of the invention. In some embodiments, phospholipids having a carbon chain length of between 4 and 24 are used. Lecithin, which also can be used, is a natural mixture of phospholipids typically derived from chicken eggs or sheep's wool. Phospholipids can be purchased from Avanti lipids (Alabastar, Ala., USA), and lipoid LLC (Newark, N.J., USA).

### Emulsifier

The compositions of the invention may comprise one or more emulsifiers. The emulsifier may be a pure emulsifying agent or a mixture of emulsifying agents. The emulsifiers of the present invention are pharmaceutically and/or immunologically acceptable. Emulsifiers generally assist in stabilizing the mixture of amphipathic compound and antigen or the mixture of amphipathic compound, antigen and adjuvant, when the mixtures are resuspended into the hydrophobic carrier.

The emulsifier may be amphipathic and therefore, the emulsifier may include a broad range of compounds. In some embodiments, the emulsifier may be a surfactant, such as for example, a non-ionic surfactant.

Examples of emulsifiers which may be used include polysorbates, which are oily liquids derived from polyethylene glycolyated sorbital, and sorbitan esters. Polysorbates may include, for example, sorbitan monooleate. Typical emulsifiers include mannide oleate (Arlacel™ A), lecithin, Tween™ 80, and Spans™ 20, 80, 83 and 85, The emulsifier is generally pre-mixed with the hydrophobic carrier.

In some embodiments, a hydrophobic carrier which already contains an emulsifier may be used. For example, a hydrophobic carrier such Montanide™ ISA-51 already contains the emulsifier mannide oleate. In other embodiments, the hydrophobic carrier may be mixed with emulsifier before combining with the amphipathic compound and antigen.

### Hydrophobic Carrier

The hydrophobic carrier may be an essentially pure hydrophobic substance or a mixture of hydrophobic substances.

Hydrophobic substances that are useful in the compositions as described herein are those that are pharmaceutically and/or immunologically acceptable. The carrier is preferably a liquid but certain hydrophobic substances that are not liquids at atmospheric temperature may be liquefied, for example by warming, and are also useful in this invention.

Oils or mixtures of oils are particularly suitable carriers for use in the present invention. Oils should be pharmaceutically and/or immunologically acceptable. Oils may be metabolizable or non-metabolizable, or a mixture of metabolizable and non-metabolizable oils may be used.

Preferred examples of oils are mineral oil (especially light or low viscosity mineral oil), vegetable oil (e.g., soybean oil), nut oil (e.g., peanut oil). A low viscosity mineral oil such as Drakeol® 6VR may be used in some embodiments. In an embodiment, the oil is a mannide oleate in mineral oil solution, commercially available as Montanide™ ISA 51. Other oils may include the Montanide ISA 700 series (Seppic Inc., France) or MAS-1 (Mercia Pharmaceuticals), for example. In embodiments where there is a pure hydrophobic carrier, the hydrophobic carrier may be mixed with emulsifier before use in the compositions of the present invention.

Animal fats and artificial hydrophobic polymeric materials, particularly those that are liquid at atmospheric temperature or that can be liquefied relatively easily, may also be used.

Liquid fluorocarbons are medically applicable hydrophobic carriers that may also be used in the practice of the invention.

### Other Components

The composition may further comprise one or more additional components such as, for example, pharmaceutically acceptable adjuvants, excipients, etc., as are known in the art: See, for example, Remington's Pharmaceutical Sciences (Remington's Pharmaceutical Sciences, Mack Publishing Company, Easton, Pa., USA 1985) and The United States Pharmacopoeia: The National Formulary (USP 24 NF19) published in 1999.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al, Immunology, 2d ed., Benjamin/Cummings: Menlo Park, C. A., 1984; see Wood and Williams, In: Nicholson, Webster and May (eds.), Textbook of Influenza, Chapter 23, pp. 317-323).

Suitable adjuvants include, but are not limited to, alum, other compounds of aluminum, Bacillus of Calmette and Guerin (BCG), TiterMax®, Ribi®, incomplete Freund's adjuvant (IFA), saponin, surface active substances such as lysolecithin, pluronic polyols, polyanions, peptides, Corynebacteriumparvum, QS-21, Freund's Complete Adjuvant (FCA), adjuvants of the TLR agonist family such as CpG, polyIC (a double stranded RNA), falgellin, lipopeptides, peptidoglycans, imidazoquinolines, single stranded RNA, lipopolysaccharides (LPS), heat shock proteins (HSP), and ceramides and derivatives such as alpha Gal-cer. Suitable Adjuvants also include cytokines or chemokines in their polypeptide or DNA coding forms such as, but not limited to, GM-CSF, TNF-alpha, IFN-gamma, IL-2, IL-12, IL-15, IL-21.

The hydrophobic carrier, discussed above, may in some instances function as an adjuvant.

The amount of adjuvant used depends on the amount of antigen and on the type of adjuvant. One skilled in the art can readily determine the amount of adjuvant needed in a particular application.

The composition may also contain one or more additional polypeptides, which may be a short synthetic polypeptide such as a T helper epitope.

### Preparation of Compositions

The antigen and amphipathic compound are mixed prior to suspension in the hydrophobic carrier. Preferably, the antigen and amphipathic compound are combined in such a manner that a substantially homogeneous mixture is formed. This may be accomplished by solubilizing the antigen and/or the amphipathic compound in a suitable solvent before the components are combined. Alternatively, the two entities can be mixed together in their dry form (e.g. by milling). In this context, a “substantially homogeneous mixture” of the antigen and amphipathic compound is a mixture in which the amphipathic compound is substantially evenly dispersed within the antigen component.

The amphipathic compound or mixture of amphipathic compounds are present in the compositions of the invention in a sufficient amount that the antigen may be resuspended in the hydrophobic carrier. The amount of amphipathic compound in the compositions of the present invention may be, for example, from about 0.1 mg to about 250 mg of amphipathic compound per ml of the composition, more preferably about 0.1 to about 120 mg of amphipathic compound per ml of the composition.

If the antigen and/or amphipathic compound are solubilized, the skilled artisan can readily identify suitable solvents or solvent systems (generally organic solvents) for solubilizing a particular amphipathic compound or antigen. In the case of an amphipathic compound that is a phospholipid, a polar protic solvent such as an alcohol (e.g. tert-butanol, n-butanol, isopropanol, n-propanol, ethanol or methanol), water, acetic acid or formic acid, or chloroform may be used. Antigens such as polypeptides may be solubilized with a polar aprotic solvent such as dimethyl sulfoxide (DMSO), dimethyl formamide (DMF), or tetrahydrofuran (THF). Other solvents, such as non-polar solvents (e.g. hexane) may be used, as well as liquid CO₂.

In some cases, the same solvent can be used to solubilize both the amphipathic and the antigen of interest.

The solubilized antigen and solubilized amphipathic compound are then mixed. Alternatively, the antigen and amphipathic compound may be mixed prior to solubilization, and then solubilized together. In a further alternative, only one of the amphipathic compound or the antigen is solubilized, and the non-solubilized component added.

Solvent is then removed, and this may be accomplished using standard techniques. If a readily evaporated solvent is used, such as ethanol, methanol, or chloroform, a standard evaporation technique, such as rotary evaporation, evaporation under reduced pressure, or freeze drying may be employed. Solvents, such as water, may be removed by e.g. lyophilization, freeze drying or spray drying. Low heat drying that does not compromise the integrity of the components can also be used. Heat can also be used to assist in resuspending the antigen/amphipathic compound mixture prior to its use.

Preferably, solubilized antigen and solubilized amphipathic compound are mixed thoroughly and then dried as described above. Preferably, the dried mixture is a substantially homogeneous mixture of antigen and amphipathic compound wherein the amphipathic compound is substantially evenly dispersed within the antigen component. If instead the substantially homogeneous mixture of antigen and amphipathic compound is formed by a solvent-free process such as dry milling, there is of course no need for a drying step.

The dry mixture of antigen and amphipathic compound is then re-suspended in the hydrophobic carrier to provide the finished composition. In some embodiments, the hydrophobic carrier may contain an emulsifier, as described in detail above, which is provided in an amount sufficient to re-suspend the dry mixture of antigen and amphipathic compound in the hydrophobic carrier and maintaining the antigen and amphipathic compound in suspension in the hydrophobic carrier. For example, the emulsifier may be present at about 5% to about 15% weight/weight or weight/volume of the hydrophobic carrier.

Additional components as described above, such as an adjuvant or other pharmaceutically acceptable auxiliaries, may be added at any stage in the formulation process. For instance, one or more such additional components may be combined with the antigen or amphipathic compound either before or after solubilization, or added to the solubilized mixture. Additional components, such as an adjuvant, may instead be added to or combined with the dried mixture of antigen and amphipathic compound, or combined with the hydrophobic carrier either before or after suspension of the dry mixture of antigen and amphipathic compound in the hydrophobic carrier. Similarly, if a dry mixing technique is used, any additional components may be added either before or after milling.

Unexpectedly, a strong immune response is obtainable when the antigen is suspended in the hydrophobic carrier as described, in the absence of substantial quantities of water. It is not expected that antigens could be placed in a hydrophobic carrier unless formulated in the composition described herein. In practice, it may be difficult to obtain completely water-free compositions. That is, although all or substantially all water is removed, such as by evaporation, lyophilization or any other suitable drying technique, at the appropriate stage in the formulation process, there may be small amounts of water remaining. For example, individual components of the composition may have bound water that may not be completely removed by processes such as lyophilization or evaporation and certain hydrophobic carriers may contain small amounts of water dissolved therein. When water is present, for example, in the form of an emulsion in the composition, it is expected that some amount of antigen may become partitioned into the water. Accordingly, the presence of water in the composition decreases the amount of antigen suspended in the hydrophobic carrier and thus, water is undesirable in the final composition.

The finished composition is substantially free of water. By “substantially free of water” is meant that the proportion of antigen suspended in (e.g. dissolved in) water relative to the total amount of antigen in the composition (weight/weight) is low enough that the quantity of antigen suspended in water by itself is incapable of mounting an equivalent immune response to that provided by the composition as a whole. In contrast, the quantity of antigen that is suspended in the hydrophobic carrier is sufficiently high that an equivalent immune response can be generated with that quantity (i.e. the total quantity minus the antigen quantity present in a residual water component). The efficacy of the composition (i.e. its ability to produce the desired biological response) should therefore be attributed primarily to antigen suspended in the hydrophobic carrier. In this regard, the efficacy of the composition is at least 80%, 85%, 90% or 95% as great as induced by the same composition in which none of the antigen is present in a residual water component.

The compositions of the invention that are “substantially free of water” generally contain less than about 10%, 9%, 8%, 7%, 6%, 5%, 4%, 3%, 2%, 1%, 0.5%, 0.1%, 0.05% or 0.01% water on a weight/weight basis. The quantity of antigen suspended in the residual water component is expected to be less than 20%, 15%, 10%, 9%, 8%, 7%, 6%, 5%, 4%, 3%, 2%, 1% or less of the total amount (weight basis) of antigen in the composition.

Typically, the compositions of the invention are sufficiently free of water that no water-in-oil emulsion that is visible to the naked eye is formed. For instance, the presence of an undesirable water-in-oil emulsion might be detected by a non-transparent, cloudy or opaque appearance to the composition. In contrast, compositions of the invention typically have a clear or transparent appearance and are free of visible particulate matter, such as precipitated or aggregated antigen that is not suspended in the hydrophobic carrier.

The compositions as described herein may be formulated in any form suitable for delivery of an antigen to a subject, including, by way of non-limiting examples, a form that is suitable for oral, nasal, rectal or parenteral administration. Parenteral administration includes, without limitation, intravenous, intraperitoneal, intradermal, subcutaneous, intramuscular, transepithelial, intrapulmonary, intrathecal, and topical modes of administration.

### Kits and Reagents

The present invention is optionally provided to a user as a kit. For example, a kit of the invention contains one or more of the compositions of the invention. The kit can further comprise one or more additional reagents, packaging material, containers for holding the components of the kit, and an instruction set or user manual detailing preferred methods of using the kit components for a desired purpose.

In an embodiment, compositions of the invention may be provided in which the dry mixture of the antigen and amphipathic compound is packaged in a first container and the hydrophobic carrier is packaged in a second container.

The dry mixture of antigen and amphipathic compound may then be suspended in the hydrophobic carrier shortly before administration to a subject.

### Uses

The invention finds application in any instance in which it is desired to administer an antigen to a subject. The subject may be a vertebrate, such as a fish, bird or mammal, preferably a human.

In some embodiments, the compositions of the invention may be administered to a subject in order to raise an antibody response to the antigen.

An “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g. of the IgA and IgM classes) may also assemble into oligomeric forms with each other and additional polypeptide chains, for example as IgM pentamers in association with the J-chain polypeptide.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

As used herein, the term “antibody response” refers to an increase in the amount of antigen-specific antibodies in the body of a subject in response to introduction of the antigen into the body of the subject.

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

In some embodiments, the compositions of the invention may be administered to a subject in order to raise a cell-mediated immune response to the antigen. As used herein, the term “cell-mediated immune response” refers to an increase in the amount of antigen-specific cytotoxic T-lymphocytes, macrophages, natural killer cells, or cytokines in the body of a subject in response to introduction of the antigen into the body of the subject.

Historically, the immune system was separated into two branches: humoral immunity, for which the protective function of immunization could be found in the humor (cell-free bodily fluid or serum that contain antibodies) and cellular immunity, for which the protective function of immunization was associated with cells. Cell-mediated immunity is an immune response that involves the activation of macrophages, natural killer cells (NK), antigen-specific cytotoxic T-lymphocytes, and the release of various cytokines in response to a ‘non-self’ antigen. Cellular immunity is an important component of adaptive immune response and following recognition of antigen by cells through their interaction with antigen-presenting cells such as dendritic cells, B lymphocytes and to a lesser extent, macrophages, protects the body by various mechanisms such as:

1. activating antigen-specific cytotoxic T-lymphocytes that are able to induce apoptosis in body cells displaying epitopes of foreign antigen on their surface, such as virus-infected cells, cells with intracellular bacteria, and cancer cells displaying tumor antigens;
2. activating macrophages and natural killer cells, enabling them to destroy intracellular pathogens; and
3. stimulating cells to secrete a variety of cytokines that influence the function of other cells involved in adaptive immune responses and innate immune responses.

Cell-mediated immunity is most effective in removing virus-infected cells, but also participates in defending against fungi, protozoans, cancers, and intracellular bacteria. It also plays a major role in transplant rejection.

Detection of cell mediated immune response following vaccination

Since cell mediated immunity involves the participation of various cell types and is mediated by different mechanisms, several methods could be used to demonstrate the induction of immunity following vaccination. These could be broadly classified into detection of:

i) specific antigen presenting cells; ii) specific effector cells and their functions and iii) release of soluble mediators such as cytokines.
i) Antigen presenting cells: Dendritic cells and B-cells (and to a lesser extent macrophages) are equipped with special immuno-stimulatory receptors that allow for enhanced activation of T cells, and are termed professional antigen presenting cells (APC). These immuno-stimulatory molecules (also called as co-stimulatory molecules) are up-regulated on these cells following infection or vaccination, during the process of antigen presentation to effector cells such as CD4 and CD8 cytotoxic T cells. Such co-stimulatory molecules (such as CD80, CD86, MHC class I or MHC class II) can be detected by using flow cytometry with fluorochrome-conjugated antibodies directed against these molecules along with antibodies that specifically identify APC (such as CD11c for dendritic cells).
ii) Cytotoxic T cells: (also known as Tc, killer T cell, or cytotoxic T-lymphocyte (CTL)) are a sub-group of T cells which induce the death of cells that are infected with viruses (and other pathogens), or expressing tumor antigens http://en.wikipedia.org/wiki/Image:Cytotoxic T cell .jpg These CTLs directly attack other cells carrying certain foreign or abnormal molecules on their surface. The ability of such cellular cytotoxicity can be detected using in vitro cytolytic assays (chromium release assay). Thus, induction of adaptive cellular immunity can be demonstrated by the presence of such cytotoxic T cells, wherein, when antigen loaded target cells are lysed by specific CTLs that are generated in vivo following vaccination or infection.

Naive cytotoxic T cells are activated when their T-cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed”-effector cells. Activated CTL will then travel throughout the body in search of cells bearing that unique MHC Class I+peptide. This could be used to identify such CTLs in vitro by using peptide-MHC Class I tetramers in flow cytometric assays.

When exposed to these infected or dysfunctional somatic cells, effector CTL release perforin and granulysin: cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cellular immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-g, quantitative measurement of IFN-g-producing CD8 cells can be achieved by ELISPOT and by flowcytometric measurement of intracellular IFN-g in these cells.

CD4+ “helper” T-cells: CD4+ lymphocytes, or helper T cells, are immune response mediators, and play an important role in establishing and maximizing the capabilities of the adaptive immune response. These cells have no cytotoxic or phagocytic activity; and cannot kill infected cells or clear pathogens, but, in essence “manage” the immune response, by directing other cells to perform these tasks. Two types of effector CD4+ T helper cell responses can be induced by a professional APC, designated Th1 and Th2, each designed to eliminate different types of pathogens.

Helper T cells express T-cell receptors (TCR) that recognize antigen bound to Class II MHC molecules. The activation of a naive helper T-cell causes it to release cytokines, which influences the activity of many cell types, including the APC that activated it. Helper T-cells require a much milder activation stimulus than cytotoxic T-cells. Helper T-cells can provide extra signals that “help” activate cytotoxic cells. Two types of effector CD4+ T helper cell responses can be induced by a professional APC, designated Th1 and Th2, each designed to eliminate different types of pathogens. The measure of cytokines associated with Th1 or Th2 responses will give a measure of successful vaccination. This can be achieved by specific ELISA designed for Th1-cytokines such as IFN-g, IL-2, IL-12, TNF-a and others, or Th2-cytokines such as IL-4, IL-5, IL10 among others.

iii) Measurement of cytokines: released from regional lymph nodes gives a good indication of successful immunization. As a result of antigen presentation and maturation of APC and immune effector cells such as CD4 and CD8 T cells, several cytokines are released by lymph node cells. By culturing these LNC in vitro in the presence of antigen, antigen-specific immune response can be detected by measuring release if certain important cytokines such as IFN-g, IL-2, IL-12, TNF-a and GM-CSF. This could be done by ELISA using culture supernatants and recombinant cytokines as standards.

The invention finds broad application in the prevention and treatment of any disease susceptible to prevention and/or treatment by way of administration of an antigen. Representative applications of the invention include cancer treatment and prevention, gene therapy, adjuvant therapy, infectious disease treatment and prevention, allergy treatment and prevention, autoimmune disease treatment and prevention, neuron-degenerative disease treatment, and artheriosclerosis treatment, drug dependence treatment and prevention, hormone control for disease treatment and prevention, control of a biological process for the purpose of contraception.

Prevention or treatment of disease includes obtaining beneficial or desired results, including clinical results. Beneficial or desired clinical results can include, but are not limited to, alleviation or amelioration of one or more symptoms or conditions, diminishment of extent of disease, stabilisation of the state of disease, prevention of development of disease, prevention of spread of disease, delay or slowing of disease progression, delay or slowing of disease onset, conferring protective immunity against a disease-causing agent and amelioration or palliation of the disease state. Prevention or treatment can also mean prolonging survival of a patient beyond that expected in the absence of treatment and can also mean inhibiting the progression of disease temporarily, although more preferably, it involves preventing the occurrence of disease such as by preventing infection in a subject.

The skilled artisan can determine suitable treatment regimes, routes of administration, dosages, etc., for any particular application in order to achieve the desired result. Factors that may be taken into account include, e.g.: the nature of the antigen; the disease state to be prevented or treated; the age, physical condition, body weight, sex and diet of the subject; and other clinical factors.

The invention is further illustrated by the following non-limiting examples.

### Example 1

Pathogen free, female C57BL/6 mice, 6-8 weeks of age, were obtained from Charles River Laboratories (St Constant, Quebec, Canada) and were housed according to institutional guideline with water and food ad libitum, under filter controlled air circulation.

The C3 cell line used in this study, a well-described mouse model for pre-clinical cervical cancer research, is an HPV 16-expressing C3 tumor cells derived from B6 mouse embryo cells (B6mec) and transformed with the complete HPV 16 genome under its own promoter and an activated-ras oncogene. The C3 cell line develops tumors when injected subcutaneously and has been used in cancer challenge studies to examine the efficacy of vaccine administered before or after C3 tumor cell implantation. The C3 cell line was maintained in Iscove Modified Dulbecco's Medium (IMDM; Sigma, St. Louis, Mo.) supplemented with 10% heat-inactivated fetal calf serum (Sigma, St. Louis, Mo.), 2 mM l-glutamine, 50 mM 2-mercaptoethanol, penicillin and streptomycin. Cells were incubated at 37 degrees Celsius/5% CO₂.

The HPV 16 E7 (H-2 Db) peptide RAHYNIVTF49-57 (SEQ ID NO: 1) containing a CTL epitope was fused to PADRE containing a CD4+ helper epitope by Dalton Chemical Laboratories Inc. (Toronto, Ontario, Canada). This peptide is hereafter designated FP and was used as antigen in vaccine at 50 micrograms/100 microliters dose. FP has been used in vaccine studies to prevent or eliminate C3 tumors in mice.

To formulate vaccines described herein, dioleoyl phosphatidylcholine (DOPC) was solubilized in tert-butanol. FP was first solubilized in dimethyl sulfoxide, although a water suspension of FP can also be used. FP was then added to the DOPC/tert-butanol mixture. Where indicated, a synthetic lipopeptide-based immune stimulating compound (the adjuvant) was resuspended in water and added to the DOPC/FP/tert-butanol mixture. A dry homogenous mixture of antigen (with or without adjuvant) was prepared by removing the solvent and water present in the formulation by lyophilization.

The dry mixture was then suspended in Incomplete Freund's adjuvant, a mineral oil-based model hydrophobic carrier.

The efficacy of these formulations was compared to those consisting of antigen (with or without adjuvant) in typical water-in-oil emulsion such as an incomplete Freund's adjuvant-based emulsion consisting primarily of water (containing the antigen/adjuvant) in a continuous mineral-oil based oil carrier.

To test the efficacy of these water-free formulations, groups of mice (7 to 10 mice per group) were injected with half a million C3 cells subcutaneously in the left flank above the base of the tail. Eight days post-implantation, all mice were injected with vaccine formulations (100 microliters per dose) subcutaneously in the right flank. Five groups of mice were vaccinated as follows: Group 1 mice served as control mice and were injected with phosphate buffered saline to allow the normal progression of tumors; group 2 mice were vaccinated with a standard water-in-oil mineral oil-based emulsion (incomplete Freund's adjuvant) containing FP antigen (50 micrograms per dose) in the aqueous component of the emulsion; group 3 mice were vaccinated with a homogeneous water-free formulation prepared as described above and containing FP antigen (50 micrograms per dose), DOPC (12 micrograms per dose) and 100 microliters of a hydrophobic carrier (Incomplete freund's adjuvant); group 4 mice were vaccinated with a standard water-in-oil mineral oil-based emulsion (incomplete Freund's adjuvant) containing FP antigen (50 micrograms per dose) and Pam3Cys adjuvant (50 micrograms per dose) in the aqueous component of the emulsion; group 5 mice were vaccinated with a homogeneous water-free formulation prepared as described above and containing FP antigen (50 micrograms per dose), Pam3Cys adjuvant (50 micrograms per dose), DOPC (12 micrograms per dose) and 100 microliters of a hydrophobic carrier (Incomplete Freund's adjuvant); tumor growth was monitored in all mice on a weekly basis for 42 days to assess the effect of vaccination on tumor growth.

All mice in group 1 implanted with C3 cells developed tumors with tumors reaching 1881 cubic millimeters in size on day 42 post tumor implantation. Control vaccinations consisting of FP antigen in a water-in-oil emulsion controlled tumor growth in mice (group 2), with tumors progressing to an average size of 548 cubic millimeters on day 42. Group 3 mice vaccinated with a water-free composition of FP antigen in a hydrophobic carrier and utilizing DOPC as an amphipathic carrier to ensure homogenous resuspension of the antigen in the oil, was significantly more efficacious than group 2 vaccines in controlling tumor growth. Average tumor size in group 3 mice was 73 cubic millimeter on day 42 post tumor implantation, with 6 out of 8 mice being rendered tumor free. The addition of an adjuvant, Pam3Cys in this example, improved efficacy of the water-in-oil vaccine formulation used for group 2 slightly (group 4 average tumor size on day 42 was 320 cubic millimeters versus group 2 average tumor size of 548 cubic millimeters). The addition of Pam3Cys adjuvant improved the efficacy of the water-free vaccine formulation further (group 5 vaccine), with an average tumor size of 14 cubic millimeters on day 42, with 6 out of 7 mice being rendered tumor-free.

These results clearly show that the exclusion of water from vaccine formulations and the use of a phospholipid based amphipathic molecule to ensure the homogeneity of immune activating compounds in the hydrophobic carrier, generate enhanced targeted immune responses.

### Example 2

Pathogen free, female C57BL/6 mice, 6-8 weeks of age, were obtained from Charles River Laboratories (St Constant, Quebec, Canada) and were housed according to institutional guideline with water and food ad libitum, under filter controlled air circulation.

The HPV 16 E7 (H-2 Db) peptide RAHYNIVTF49-57 (SEQ ID NO: 1) containing a CTL epitope was fused to PADRE containing a CD4+ helper epitope by Dalton Chemical Laboratories Inc. (Toronto, Ontario, Canada). This peptide is hereafter designated FP and was used as antigen in vaccine at 20 micrograms/100 microliters dose.

Vaccine efficacy was assessed by Enzyme-linked Immunospot assay (ELISPOT), a method that allows the ex vivo detection of antigen-specific cellular immune responses in splenocytes harvested from immunized C57BL/6 mice. The ELISPOT assay is useful for assessing the presence/absence of an antigen-specific immune response but has its limitations when used as a correlate of vaccine efficacy against a target in vivo. Briefly, on day 8 post-immunization, a 96-well nitrocellulose plate was coated with capture antibody, a purified anti-mouse IFN-gamma antibody, by incubation overnight at 4° C., then blocked with complete media. Splenocytes were added to wells at an initial concentration of 5×105 cells/well in a volume of 100 μl and a row of serial dilutions prepared. Cells in a dilution series were stimulated with the specific peptide RAHYNIVTF49-57 (10 μg/ml). The plate was incubated overnight at 37° C./5% CO₂. Next day, the plate was incubated with detection antibody (a biotinylated anti-mouse IFN-γ antibody), for 2 hours at room temperature. Unbound detection antibody was removed by washing and the enzyme conjugate (Streptavidin-HRP) was added. Following 1 hour incubation at room temperature, unbound enzyme conjugate was removed by washing and the plate was stained with an AEC substrate solution for 20 minutes. The plate was washed, allowed to air dry overnight, and foci of staining were counted using a magnifying lens.

To formulate vaccine described herein, dioleoyl phosphatidylcholine (DOPC) was solubilized in tert-butanol. FP was first solubilized in dimethyl sulfoxide, although a water suspension of FP can also be used. FP was then added to the DOPC/tert-butanol mixture. A dry homogenous mixture of antigen and DOPC was prepared by removing the solvent present in the formulation by lyophilization.

The dry mixture was then suspended in Incomplete Freund's adjuvant, a mineral oil-based model hydrophobic carrier.

The efficacy of this formulation was compared to one consisting of antigen in typical water-in-oil emulsion such as an incomplete Freund's adjuvant-based emulsion consisting primarily of water (containing the antigen) in a continuous mineral-oil based oil carrier. The efficacy of the formulation was also compared to one consisting the antigen diluted directly from a antigen/dimethyl sulfoxide stock solution into typical water-in-oil emulsion such as an incomplete Freund's adjuvant-based emulsion.

To test the efficacy of this water-free formulation, groups of mice (4 mice per group) were injected with vaccine formulations (100 microliters per dose) subcutaneously in the right flank. Three groups of mice were vaccinated as follows: Group 1 mice served as control mice and were vaccinated with a standard water-in-oil mineral oil-based emulsion (incomplete Freund's adjuvant) containing FP antigen (20 micrograms per dose) in the aqueous component of the emulsion; group 2 mice were vaccinated with a homogeneous water-free formulation prepared as described above and containing FP antigen (20 micrograms per dose), DOPC (12 micrograms per dose) and 100 microliters of a hydrophobic carrier (Incomplete freund's adjuvant); group 3 mice served as control mice and were vaccinated with a homogeneous water-free formulation lacking the amphipathic carrier (DOPC) but containing FP antigen (20 micrograms per dose), and 100 microliters of a hydrophobic carrier (Incomplete freund's adjuvant). Spleens were removed from all mice 8 days later to examine the presence of an antigen-specific immune response by the ELISPOT assay.

Group 1 mice generated a significant antigen-specific cellular response. The presence of such a response as detected by ELISPOT indicates that the vaccine formulation has the potential to induce an effective immune response against a target in vivo. Group 2 mice that were injected with the water-free formulation containing FP, the amphipathic carrier (DOPC), and the hydrophobic oil carrier were also capable of inducing an immune response that indicates a potential to induce an effective immune response against a target in vivo. Group 3 mice that were injected with a water-free formulation lacking the amphipathic carrier (DOPC) but containing FP and the hydrophobic oil carrier induced a significantly reduced immune response as detected by ELISPOT, clearly indicating that this particular formulation has a considerably lower immunogenic potential. These results clearly show that the exclusion of water from vaccine formulations has the potential to generate enhanced targeted immune responses, provided that a phospholipid-based amphipathic molecule is present in the formulation.

### Example 3

In this example, poly IC double stranded RNA (Pierce, Milwaukee, USA) was used as a representative molecule that has physical and chemical characteristics that are similar to those of a genetic antigen construct (a nucleotide based plasmid or RNA molecule). PolyIC also serves as a representative of nucleotide based adjuvants that may be co-formulated with antigen in the invention. To formulate 1 ml final volume of vaccine described herein, 120.0 mg of dioleoyl phosphatidylcholine (DOPC) was solubilized in 480 ul of tert-butanol at a temperature of 40° C. for 10 to 15 minutes with shaking. polyI:C was first solubilized in water at a concentration of 5 mg/ml. Then 80 ul of polyI:C (0.4 mg) were further diluted in 320 ul of water. The polyI:C dilution was then added and mixed to the DOPC/tert-butanol mixture in vial 21. A dry homogenous mixture of DOPC/adjuvant was prepared by removing the solvent and water present in the formulation by lyophilization. A control formulation containing polyIC alone (vial 26) was made in the same manner as described above, with the exception that DOPC was not added to tert butanol.

The dry contents of vials 21 and 26 were then suspended by adding 0.88 ml of a hydrophobic carrier to vial 21, and 1 ml of hydrophobic carrier to vial 26. The hydrophobic carrier used was a mineral oil containing mannide oleate and known as Montanide® ISA 51 (Seppic, France). The dry mixture was resuspended in the hydrophobic carrier by vortexing for approximately 3 minutes (vial 21), or a minimum of 30 minutes (vial 26). Vials 21 and 26 were compared by visual inspection along side a vial containing 1 ml of hydrophobic carrier alone (vial labelled ISA51).

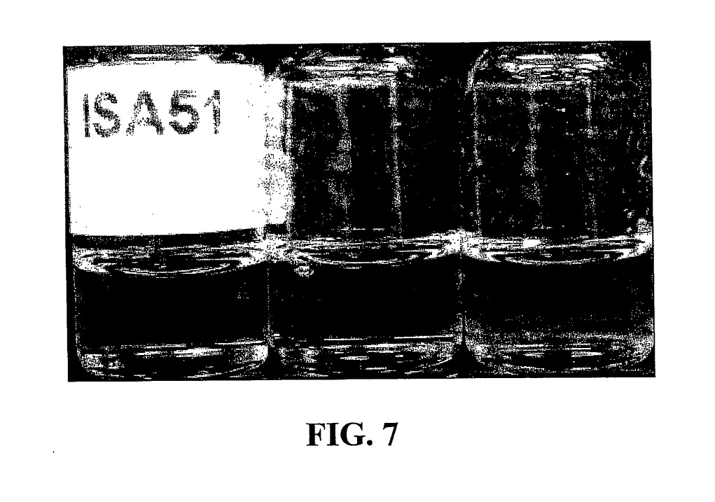

Upon visual inspection (FIG. 7), the contents of vial 21 appeared similar to that of ISA51, with no visible particulate material present. This suggested that the nucleotide molecules were effectively suspended in the hydrophobic carrier in the presence of DOPC. In contrast, vial 26 lacking DOPC and containing only the nucleotide molecules and hydrophobic carrier, contained a heterogenous suspension of nucleotide molecules in the hydrophobic carrier that can be easily detected by visual inspection. In the absence of DOPC, the hydrophilic nucleotide molecules could not be suspended in the hydrophobic carrier. This clearly demonstrates that a molecule such as DOPC that is amphipathic in nature, facilitated the formulation of hydrophilic nucleotide-based molecules in a hydrophobic carrier in the absence of a significant quantity of water.

### Example 4

In this example, peptide S9L (SVYDFFVWL) (SEQ ID NO: 2) and F21E (FNNFTVSFWLRVPKVSASHLE) (SEQ ID NO: 3) were used as model antigens. Peptides were synthesized chemically by a custom peptide manufacturer (Anaspec, San Jose, USA). To formulate 1 ml final volume of these peptides in the invention described herein, 120.0 mg of dioleoyl phosphatidylcholine (DOPC) was solubilized in 470 ul of tert-butanol at a temperature of 40° C. for 10 to 15 minutes with shaking. Then the mixture was cooled down to room temperature (22° C.-25° C.). S9L and F21E were first solubilized separately in dimethyl sulfoxide at a concentration of 5 mg/ml. Then 10 ul of S9L (50 ug) and 10 ul F21E (50 ug) were added sequentially while vortexing to the DOPC/tert-butanol mixture. To complete the mixture volume, 400 ul of water were added and mixed to the S9L/F21E/DOPC/tert-butanol mixture in vial 30. A dry homogenous mixture of DOPC/peptides was prepared by removing the solvent and water present in the formulation by lyophilization. A control formulation containing S91, and F21E peptides (vial 35) was made in the same manner as described above, with the exception that DOPC was not added to tert butanol.

The dry contents of vials 30 and 35 were then suspended by adding 0.88 ml of a hydrophobic carrier to vial 30, and 1 ml of hydrophobic carrier to vial 35. The hydrophobic carrier used was a mineral oil containing mannide oleate and known as Montanide® ISA 51 (Seppic, France). The dry mixture was resuspended in the hydrophobic carrier by vortexing for approximately 30 seconds (vial 30), or a minimum of 30 minutes (vial 35). Vials 30 and 35 were compared by visual inspection along side a vial containing 1 ml of hydrophobic carrier alone (vial labelled ISA51).

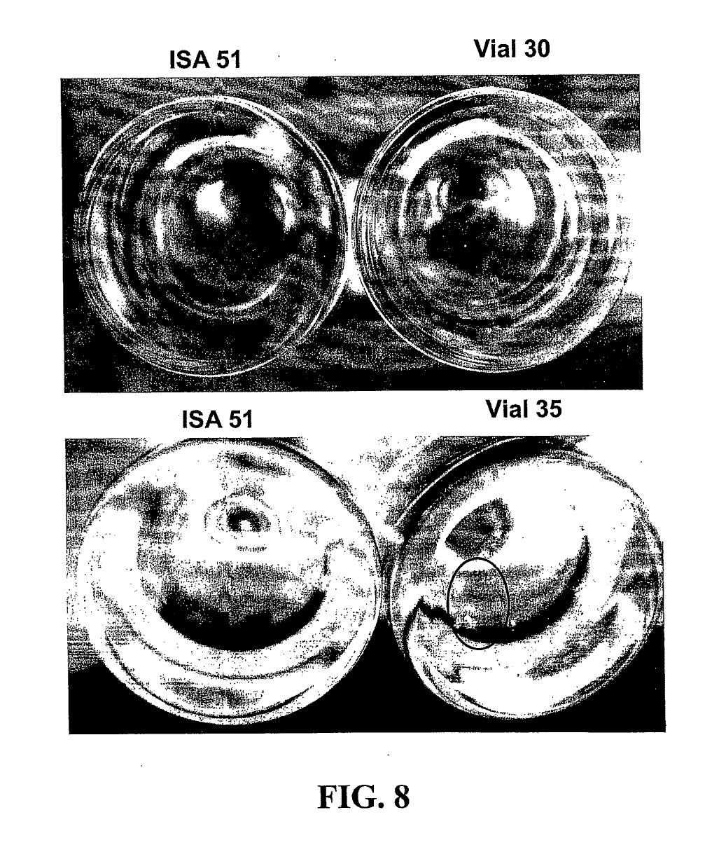

Upon visual inspection (FIG. 8), the contents of vial 30 appeared similar to that of ISA51, with no visible particulate material present. This suggested that the peptides were effectively suspended in the hydrophobic carrier in the presence of DOPC. In contrast, vial 35 lacking DOPC and containing only the peptides and hydrophobic carrier, contained a heterogeneous suspension of peptide aggregates in the hydrophobic carrier that can be easily detected by visual inspection. In the absence of DOPC, peptide-based antigens could not be suspended in the hydrophobic carrier. This clearly demonstrates that a molecule such as DOPC that is amphipathic in nature, facilitated the formulation of peptide based antigens in a hydrophobic carrier in the absence of a significant quantity of water.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an”, and “the” include plural reference unless the context clearly dictates otherwise. Unless defined otherwise all technical and scientific terms used herein have the same meaning as commonly understood to one of ordinary skill in the art to which this invention belongs.

As used herein and in the claims, the transitional term “comprising”, is intended to be synonymous with “including” or “containing” and is inclusive or open-ended and does not exclude additional, unrecited elements or method steps. The transitional phrase “consisting of” is intended to exclude any element, step, or ingredient not specified. The transitional phrase “consisting essentially of” is intended to limit the scope of a claim to the specified materials or steps and those that do not materially affect the basic and novel characteristic(s) of the claimed invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A composition comprising:
an antigen;
an amphipathic compound; and
a hydrophobic carrier;
wherein the composition is substantially free of water.

2. The composition according to claim 1, comprising less than 10% water by weight, based on the total weight of the composition.

3. The composition according to claim 1, wherein less than 20% of the antigen (by weight) is suspended in water.

4. The composition according to claim 1, wherein the hydrophobic carrier comprises an emulsifier.

5. The composition according to claim 1, wherein said amphipathic compound comprises a phospholipid.

6. The composition according to claim 1, wherein said amphipathic compound is lecithin.

7. The composition according to claim 1, wherein said hydrophobic carrier comprises an oil or a mixture of oils.

8. The composition according to claim 4, wherein said emulsifier comprises Span80, mannide oleate or a mixture thereof.

9. The composition according to claim 1, wherein said antigen comprises: a polypeptide, a polynucleotide encoding a polypeptide, a peptide, a live bacterium, a killed bacterium, a live virus, an attenuated virus, an inactivated bacterium, or a part thereof.

10. The composition according to claim 1, further comprising an adjuvant.

11. The composition according to claim 1, comprising about 0.1 to about 250 mg of the amphipathic compound per ml of the composition.

12. The composition according to claim 4, wherein the hydrophobic carrier comprises about 5% to about 15% by weight of the emulsifier.

13. The composition according to claim 1, wherein said antigen and said amphipathic compound are provided as a dry mixture in a first container, and said hydrophobic carrier and emulsifier are provided in a second container.

14. A process for making the composition according to claim 1, the process comprising:
(a) combining an antigen and an amphipathic compound to form a dry mixture; and
(b) suspending said mixture in a hydrophobic carrier.

15. The process according to claim 14, further comprising adding an adjuvant to the composition.

16. The process according to claim 14, wherein said hydrophobic carrier comprises an emulsifier.

17. The process according to claim 14, wherein said combining step comprises:
solubilizing said antigen;
solubilizing said amphipathic compound;
combining said solubilized antigen and said solubilized amphipathic compound to form a mixture; and
drying said mixture.

18. The process according to claim 14, wherein said combining step comprises dry milling said antigen with said amphipathic compound.

19. A method comprising administering the composition according to claim 1 to a subject.

20. A method for inducing an antibody response or cell-mediated immune response in a subject, said method comprising administering to a subject in need thereof the composition according to claim 1.

21. The method of claim 19, wherein the composition is administered orally, nasally, rectally or parenterally


SEQUENCE LISTING (4 sequences)

SEQ ID NO: 1 - protein, 9 aa, human papilloma virus
RAHYNIVTF

SEQ ID NO: 2 - protein, 5 aa, artificial sequence
  PADRE peptide
PADRE

SEQ ID NO: 3 - protein, 9 aa, artificial sequence
  peptide S9L
SVYDFFVWL

SEQ ID NO: 4 - protein, 21 aa, artificial sequence
  peptide F21E
FNNFTVSFWLRVPKVSASHLE